(19) United States
(12) Patent Application Publication
(10) Pub. No.: US 2009/0276053 A1
(43) Pub. Date: Nov. 5, 2009

(54) Coated Implants

(76) Inventors: Timothy Brown, Negaunee, MI (US); Qi-Bin Bao, Marquette, MI (US)

Correspondence Address: FITCH EVEN TABIN & FLANNERY, 120 SOUTH LASALLE STREET, SUITE 1600, CHICAGO, IL, 60603-3406, US

(21) Appl. No.: 12/428,313

(22) Filed: Apr. 22, 2009

Related U.S. Application Data
(60) Provisional application No. 61/047,061, filed on Apr. 22, 2008.

(51) Int. Cl. A61F 2/30

(52) U.S. Cl. 623/18.11; 623/23.57

(57) ABSTRACT

An implantable device which provides structural support for the skeletal system, such as the spine, coated with hydroxyapatite (HA). This invention provides a mechanically and bioactively functional coating of hydroxyapatite (HA) without altering the biocompatibility profile of the poly-ether-ether-ketone (PEEK) substrate.

### CROSS-REFERENCE TO RELATED APPLICATIONS

This application claims the benefit of U.S. Provisional Application Ser. No. 61/047,061, filed Apr. 22, 2008, which is hereby incorporated by reference as if reproduced in its entirety herein.

### FIELD OF THE INVENTION

This invention pertains generally to implantable medical devices and particularly to implantable medical devices for inter-vertebral fusion and/or motion preservation.

### BACKGROUND OF THE INVENTION

Many people develop back pain during the course of their life due to traumatic injury, disease, or genetic defect. Typically, the patients' discs, which support the spine, are damaged causing the vertebral discs to bulge or herniate. The disc bulge then impinges on the nerves of the spine and cause back pain. Surgeons often perform a discectomy to trim the disc bulge to alleviate back pain. However, the discectomy may structurally weaken the disc and can lead to subsequent structural failure of the disc due to wear and aging. Disc failure can cause impingement on the nerves of the spine and cause back pain once again.

Surgical insertion of a medical device to structurally support and separate the vertebras is one method for reducing debilitating back pain and allowing patients to regain normal life activities. Typically, a vertebral body replacement device (VBR) or interbody cage is inserted to support and separate the vertebrae of the patient and/or promote fusion therebetween.

The VBR or interbody cage may be inserted into the intervertebral space in an orientation where surfaces of the device with teeth formed thereon face the adjacent vertebral surfaces. Stability of the spine benefits from the VBR or interbody cage having a contour or shape that generally follows the surface shape of the endplates. As the endplates are generally slightly concave, the surface portions of the VBR including the gripping teeth often have a corresponding contour.

Many VBRs or interbody cages are composed of strong materials, such as PEEK. The purpose of fusing adjacent vertebrae by use of a VBR or interbody cage is to develop a lattice, matrix, or solid mass of bone joined with and extending between the adjacent vertebrae and through the intervertebral space. Eventually, the formed or developed bone and the vertebrae are joined to provide a somewhat unitary incompressible structure that maintains the proper spatial relationship for the size to reduce or eliminate the impingement on the spinal cord. Unfortunately, these commonly used stronger materials, such as PEEK, are not bio-resorbable, which limits the effectiveness for fusing adjacent vertebrae.

Further, the VBR or interbody cage formed of these stronger materials is unable to transubstantiate into bone, join with bone, or be absorbed by the body for replacement by bone growth. This results in a boundary interface, otherwise known as a fibrous tissue layer, or encapsulation, between the implant device and any resultant bone growth. This can be addressed by reducing the size of the VBR or interbody cage so that more graft material may be packed into the intervertebral space around the VBR or interbody cage. However, reducing the size of the VBR or interbody cage reduces the surface contact between the VBR or cage and the vertebra and may result in a less secure implantation or subsidence leading to a poor clinical outcome.

Attempts have been made to create a composite material that combines the strength of the stronger VBR or interbody cage materials with artificial bone materials such as hydroxyapatite (hereinafter HA) to reduce the boundary interface and promote improved bony adhesion. Stronger materials, such as PEEK, may be coated with HA using the plasma spray process. However, creating such a VBR or interbody cage with the proper balance of mechanical strength and bioactive functionality has been elusive. In particular, there have been issues with balancing the thickness of the hydroxyapatite so as to be thick enough to promote bony ingrowth while being thin enough to have sufficient adhesion to the PEEK implant.

Surface preparation of the polymer substrate may also alter the biocompatibility profile of the surface of the substrate leading to decreased adhesion between the HA and the substrate. Therefore, the plasma spray HA coating process has been problematic because of issues of adhesion or alteration of the substrate materials. For example, the plasma spraying of HA onto carbon fiber reinforced PEEK is disclosed in a paper entitled “Plasma-sprayed hydroxylapatite coating on carbon fiber reinforced thermoplastic composite materials”, by S. W. Ha, J. Mayer, B. Koch and E. Wintermantel, Journal of Materials Science, Materials in Medicine 5 (1994), 481-484. The authors of the paper report that the carbon fibers in the outer layers were altered during the plasma spray process. This alteration was induced by sandblasting the surface and evaporation of the PEEK matrix resulting in a low adhesion value of 2.8 MPa.

The plasma spraying process of carbon fiber composites is also disclosed in U.S. Pat. No. 5,925,458 to Vanderstraeten. The patent discloses a process whereby the substrate surface does not require any pretreatment to key or roughen the surface prior to coating. This results in adhesion strength of 17.59 MPa. However, the high adhesion strength is attributed to the molten HA particles contained in the plasma stream, which, upon contact with the composite, “melt” the surface of the composite locally and thereby become bonded into the composite. No recognition was made in the U.S. Pat. No. 5,925,458 that the melting of a composite would alter the biocompatibility profile of the composite.

Melting of the polymer surface may induce biocompatibility changes at the polymer surface, thereby rendering the coated implant useless for implantable applications. It is therefore desirable to have an implant for spinal or orthopedic applications consisting of a biocompatible polymer that has an osteoconductive surface applied to the polymer surface using the plasma spray process.

### SUMMARY OF THE INVENTION

In accordance with the present invention, a composite vertebral body replacement (VBR) or interbody cage device is provided comprising PEEK plasma coated with hydroxyapatite (HA) in which no significant alteration to the biocompatibility profile has occurred to the PEEK, particularly the outer surface of the PEEK. The HA coating has a sufficient mechanical bond with the PEEK to allow the HA to sufficiently adhere to the PEEK support structure without having to melt the PEEK to obtain sufficient bond strength. Therefore, the HA coated PEEK preserves the biocompatibility profile of the PEEK and provides a bioactive interface for improved biologic integration between the implant and patient. The HA coating provides sufficient bioactivity to allow an interface between the adjacent bone and HA to allow bone ingrowth, ongrowth, or otherwise act as an osteoconductive agent, i.e. fusion, by the patient's body.

One advantage of the present invention is the combination of mechanical bonding strength between the PEEK and HA and the bioabsorbable osteoconductive surfaces of the HA coating to instigate bone fusion. The HA coating allows the implant to be integrated into the surrounding bone tissue rather than being encapsulated in the vertebral space. Further, the proven clinical performance of PEEK and the surgeon's familiarity with tools and techniques used with PEEK implants is retained by the implant of the present invention being formed with a PEEK substrate and an HA coating.

Another advantage of the HA coated PEEK composite is the consistent size and strength of the implants, which is not available with most hydroxyapatite or bone implants. The implants can be entirely coated with HA, or, depending on the configuration of the implant, can include uncoated portions along areas which may encounter increased stress, such as tool engagement portions. Additionally, the HA coated PEEK composite is radiolucent in nature which allows subsequent x-ray films to be taken to determine when and if fusion has taken place between the implant and the adjacent bone. In contrast, pure HA implants are radio opaque and determination of the occurrence of fusion between the implant and the adjacent bone can be very difficult to determine.

Yet another advantage of the HA coated PEEK composite is the amorphous crystalline structure of the lower layer of the HA coating (i.e., the layer in constant contact with the PEEK surface), which provides a strong mechanical bond to roughened PEEK surfaces.

Additional advantages and features of the invention will become apparent for the following description and attached claims taken in combination with the accompanying drawings or figures.

### BRIEF DESCRIPTION OF THE DRAWINGS

FIG. 1 is a graph showing the exothermic peak indicating the crystalline temperature of the PEEK substrate and an endothermic peak indicating the melting temperature of the PEEK substrate as analyzed by DSC;

FIG. 2 is a graph showing the endothermic peak indicating the melting temperature of the PEEK on a second heating cycle;

FIG. 3 is a graph showing an ATR spectrum for a PEEK substrate after coating with HA indicating a uniform PEEK substrate composition;

FIGS. 4 and 5 are microscope photos of an uncoated PEEK substrate and a PEEK substrate having an HA coating showing the differences in porosity and crystalline structure;

FIG. 6 is a microscope photo of a cut cross-section of an HA coated PEEK substrate showing the interface between the PEEK substrate and the HA coating;

FIG. 7 is a perspective view of an HA Coated VBR in accordance with one aspect of the invention showing the implant body coated with HA and a tool engagement portion uncoated;

FIG. 8 is a perspective view of an HA coated VBR in accordance with another aspect of the invention showing the implant body coated with HA and a tool engagement portion uncoated;

FIGS. 9 and 9A are perspective views of an uncoated VBR and an identical HA coated VBR in accordance with another aspect of the invention;

FIGS. 10 and 10A are perspective views of an uncoated VBR and an HA coated VBR in accordance with another aspect of the invention;

FIGS. 11 and 11A are perspective views of an uncoated VBR and an HA coated VBR in accordance with another aspect of the invention;

FIGS. 12 and 12A are perspective views of an uncoated VBR and an HA coated VBR in accordance with another aspect of the invention;

FIGS. 13 and 13A are perspective views of the top surfaces of the top and bottom portions of an articulating implant showing the concave receiving portion and a piercing portion;

FIGS. 14 and 14A are perspective view of the bottom surfaces of the top and bottom portions of an articulating implant showing the ball portion and piercing portions;

FIG. 15 is a perspective view of the assembled articulating implant of FIGS. 13, 13A, 14 and 14A;

FIG. 16 is a perspective view of a of a hip replacement device;

FIG. 17 is a perspective view of a knee replacement device; and

FIG. 18 is a schematic of a plasma spray process.

### DETAILED DESCRIPTION OF THE PREFERRED EMBODIMENTS

The present invention describes the coating of poly-ether-ether-ketone (PEEK), a known biostable and biocompatible thermoplastic polymer, with hydroxyapatite (or other crystalline phases of HA) using a plasma spray process for orthopedic and spinal applications. The present invention is not limited to HA coated PEEK implants, but also includes any polymer of the poly-aryl-ether-ketone family such as, but not limited to, poly-ether-ketone (PEK) and poly-ether-ketone-ether-ketone-ketone (PEKEKK). The polymer of the present invention can be dense or have a certain porosity to enable bone ingrowth.

In order to retain the biocompatible profile of the implant, the plasma spray process either minimally or not at all thermally degrades the PEEK substrate. More particularly, the coating does not thermally degrade the polymer or affect its thermal, mechanical, or biocompatibility properties. Further, the plasma spray process has characteristics that are similar to those on metallic substrates.

In carrying out the process of HA coating PEEK implants in accordance with the present invention, the following characteristics of the coating are preferably realized:

TABLE 1
HA Coating Parameters

Crystallinity of the coating of at least about 45%, preferably
of at least about 60%, with maximum allowable levels of other
crystalline phases no more than about 5%.
Coating porosity between about 5 and 15%.
CaP ratio centered about the stoichiometric ratio of HA,
approximately 1.67.
Bond strength of the coating to the PEEK of at least about 15
MPa.
Hydroxyapatite (HA) coating thickness ranging from about 45 to
100 μm, more preferably about 45 to 80 μm.

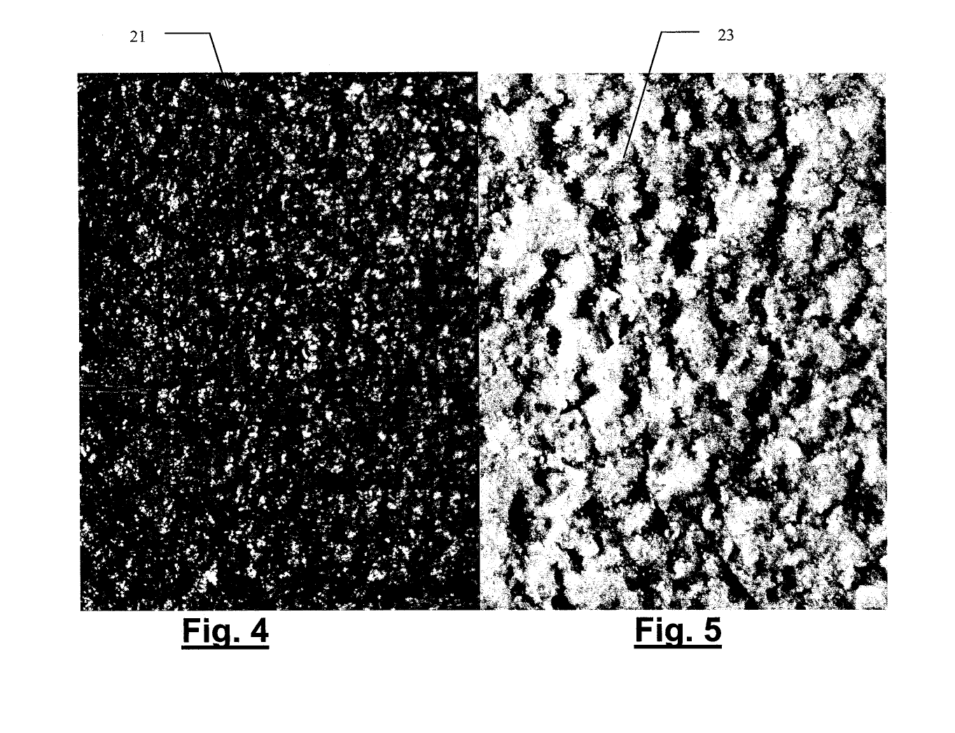

Referring to FIGS. 4 and 5, a microscopic photographic image depicts both uncoated PEEK 21 and an image of the HA coated PEEK 23. The uncoated PEEK 21 is a standard PEEK implant without surface preparations. The standard PEEK implant has limited porosity and is not bioresorbable. The HA coated PEEK 23 implant, in particular the outer surface thereof, shows significant crystallintity and porosity.

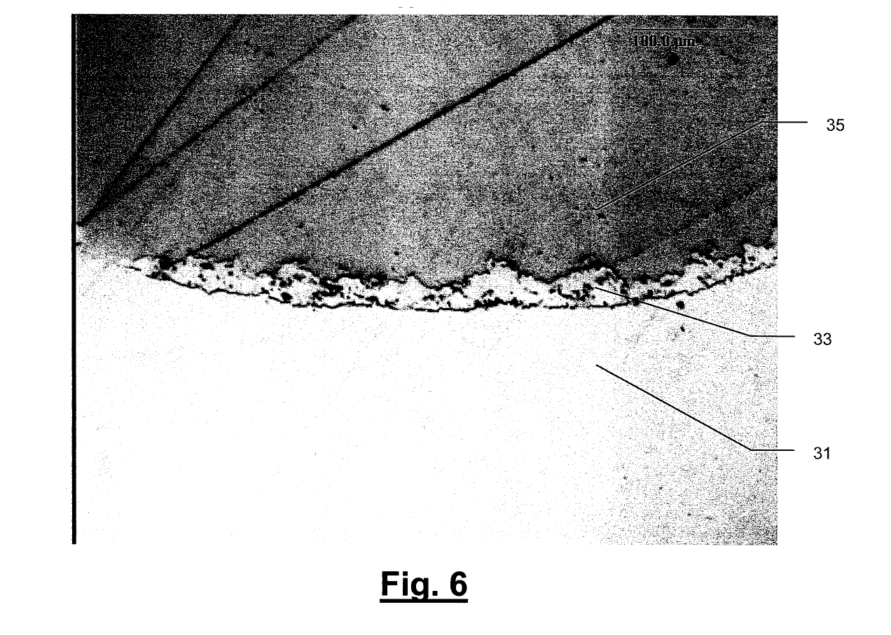

Referring to FIG. 6, a metallographic image is shown illustrating the cross-section of the HA coating on the PEEK substrate. The PEEK substrate 31 is shown on the bottom of FIG. 6. The HA coating 33 shows the depth of the entire HA coating that varies over the surface of the PEEK. A layer of epoxy resin 35 is included to highlight the contours of the HA coating 33.

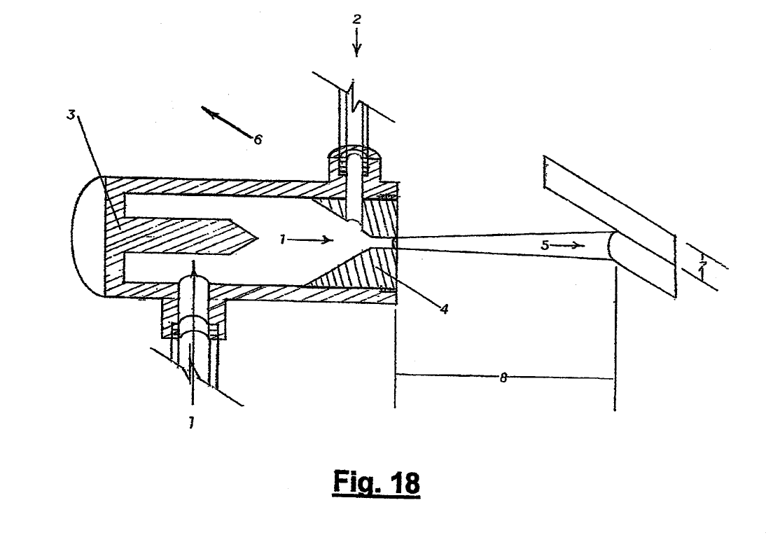

The plasma spray coating process is generally known in the arts and can be utilized to achieve an unaltered biocompatibility profile of the PEEK substrate material in accordance with the present invention. A schematic illustration of a plasma spray coating process is shown in FIG. 18.

Prior to coating PEEK (or other similar polymers) with HA (or other crystalline phases of HA), the surface of the PEEK is textured to provide a more suitable surface for HA deposition. Texturing of the surface further enhances the bond strength between the coating and the PEEK and also enhances fixation of the PEEK between the adjacent vertebrae, due to bony ingrowth, upon resorption of the coating. The surface of the PEEK may be textured into microstructured, macrostructured, microporous or macroporous morphologies. Texturing of the PEEK can be accomplished, for example, by grit blasting using a suitable media such as alumina, or by machined grooves or threads, or any other method that may allow for texturing of the surface. Alternatively, a porous coating, such as a titanium coating, may be applied to the surface. Any methodology may be utilized to texture the surface of the PEEK so long as to not illicit an adverse biological event or other affect the biocompatability, nor compromise the characteristics of the coating such as but not limited to the bond strength.

Preferably, the implant body is grit blasted with alumina to prepare the implant body for the plasma spraying process. In particular, by grit blasting the implant body surface with alumina the implant body surface is textured so that the hydroxyapatite can mechanically adhere to the PEEK implant body surface to form a HA coating along the outer surface of the implant body. Further, the implant body is grit blasted and plasma sprayed such that the mechanical adhesive body between the implant body and the HA coating is at least 15.0 MPa. For example, the implant body is grit blasted with alumina so as to texture the outer surface of the implant body with a surface roughness having an Ra of 2.0 micrometers. As compared to prior art implants, it has been found that having a rougher PEEK surface, such as an Ra of 5.0 micrometers, results in lower adhesive bonding strength than in the implant of the present invention.

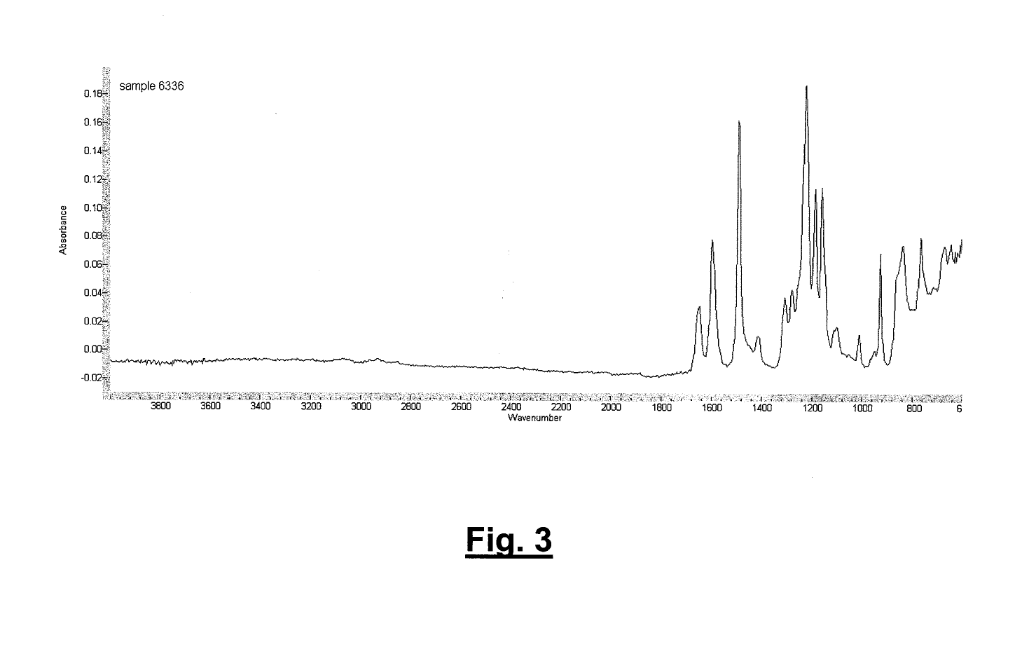

In more detail, FIG. 6 shows the uneven surface 34 of the PEEK substrate 31 along the interface with the HA coating 33. This indicates that the PEEK substrate 31 maintains the roughened or textured surface after the plasma spray process, as will be described below. The uneven surface 34 suggests that the surface 34 of the PEEK substrate was maintained at a temperature below the glass transition temperature of PEEK and that the biocompatibility of the PEEK substrate was unchanged during the HA plasma spraying process. Further, as discussed below, the biocompatibility profile of the PEEK substrate 31 at the surface 34, where the texturing takes place, is unchanged from other portions of the PEEK substrate 31, indicating the that texturing or grit blasting of the PEEK substrate 31 did not damage or affect the biocompatible qualities of the PEEK substrate 31. Further discussion of the analysis of the PEEK substrate 31 is discussed below and shown in FIG. 3.

The interface 36 between the HA coating 33 and the PEEK substrate 31 further shows the presence a well defined boundary 36 therebetween, indicating a mechanical bond, rather than a chemical bond, between the HA coating 33 and the PEEK substrate 31. By maintaining the defined boundary 36 therebetween during the plasma spray process a mechanical adhesive bond is created between the PEEK and the HA. The texturing of the PEEK substrate 31 can therefore be configured to provide a desirable PEEK outer surface 34 on which the HA is applied to achieve the increased adhesive or bonding strength between the HA coating 33 and the PEEK substrate 31.

The implant of the present invention preferably includes an HA coating thickness ranging from about 45 to about 100 micrometers, and more preferably from about 45 to about 80 micrometers. Further, the coating thickness is preferably generally uniform along the implant body surface. In addition, the implant of the present invention preferably includes a coating crystallinity of at least about 45% and adhesive mechanical bond strength between the coating and the surface of the implant of at least 15 MPa.

The coating thickness is preferably selected to accommodate the modulus of elasticity associated with the material of the implant body. In particular, an implant body comprised of PEEK has a modulus of elasticity less than titanium. Due to the brittle nature of HA, the decreased modulus of elasticity of the PEEK may cause deformation of the implant substrate and the coating, which may result in undesirable delamination of the coating. To accommodate the lower modulus of elasticity of the PEEK, a thinner HA coating, ranging from about 45 to about 100 micrometers, is selected rather than the conventional coating thicknesses of at least 150 micrometers of previous HA coated implants. As a result, the thinner HA coating is better able to withstand any deformation or change in shape of the PEEK implant during normal use.

Further, the thinner HA coating is applied so that the crystallinity of the coating is preferably at least 45%. In addition, as will be discussed further below, the pretreatment of the PEEK substrate and the process of plasma spraying the HA thereon is conducted so as to provide an adhesive mechanical bond strength of at least 15 MPa. As a result, the thinner HA coat has sufficient adhesive bonding with the PEEK substrate to resist delamination if the PEEK substrate deforms in vivo, and further the crystallinity is sufficient to promote bone ingrowth and resist delamination during the bone growth.

The process of coating PEEK with HA is similar to the plasma spray process for coating HA on to metallic substrates. The plasma spraying consists of a plasma spray gun in which an inert gas such as helium, neon, argon or nitrogen is utilized, preferably argon. The plasma gun consists of a cathode and anode between which the inert gas (or mixture of gases) passes. A high temperature direct current arc (approaching 30,000.degree. C) is generated between the cathode and anode, which ionizes the gas.

The typical feedstock powder is fully crystalline, pure HA powder, which is injected at a specified feed rate external to the gun at a specified distance and perpendicular to the plasma flow with the inert carrier gas. The hydroxyapatite powder preferably has a ratio of calcium to phosphate (Ca/P) ranging from about 1.65 to about 1.76, preferably about 1.67. Having a ratio below about 1.5 indicates the material is calcium deficient and is tricalcium phosphate rather than HA. Having a ratio above about 2.0 indicates a phosphate deficiency and tha the material is tetra calcium phosphate. In both cases, having a Ca/P ratio outside of 1.65 to 1.76 will result in a coating which is more reactive, regardless of the other properties of the coating, such as crystallinity.

Due to the high temperature of the plasma and an HA melting point temperature of 1650.degree. C, the structure and composition of the HA is significantly modified from the original feedstock powder. Other crystalline phases which may be present could be tricalcium phosphate, tetra calcium phosphate and/or oxyapatite. In addition, a substantial amorphous phase is created during the rapid cooling process. The current state of the art in coating of metallic substrates with HA ensures only trace amounts of the other crystalline phases are present and that the overall crystallinity is above 45%, with the remainder being amorphous.

The overall quality of the coating is dependent upon processing parameters and the material being coated. During the coating process, it is particularly important to not thermally degrade the PEEK or at a minimum to minimize any thermal degradation. Further, it is important that the HA coating characteristics are not significantly different from those used on metallic substrates which have a long history of clinical use. It is therefore desirable to have a coating with similar characteristics of metallic substrates while not thermally or thermal-mechanically degrading the PEEK.

In the plasma spray process, such as shown in FIG. 18, the gas flow rate 1 is controlled through the use of a mechanical control valve. The gas flow rate 1 is one factor that controls the deposition rate 5 of the hydroxyapatite. Other factors include the HA powder feed rate 2, and the discharge rate of electrons between the cathode 3 and the anode 4. The plasma spray gun traverse speed 6, which is the rate at which the entire plasma spray gun moves across the substrate, is another factor in controlling the surface temperature of the PEEK. Another control variable is the step size 7, or the distance traveled by the plasma gun from the up position and down position, each pass corresponding to an individual coat applied by the plasma spray process. Finally, cooling is also affected by the stand off distance 8, or the distance between the plasma spray gun and the implant.

The HA is plasma sprayed onto the implant body of the present invention so as to not affect the biocompatibility of the implant body. In particular, the coating is plasma sprayed so that the temperature of the outer surface of the implant body does not exceed the glass transition temperature. In this manner, the outer surface of the implant body remains in the same state or condition from before the plasma spraying process to after the plasma spraying process.

In particular, while plasma spray coating, it is advantageous to have a cooling process of cooling the PEEK surface. PEEK has a glass transition temperature of 143.degree. C, a melt temperature of 343.degree. C and a thermal conductivity of 0.25 W/mK. Titanium, the most common metallic substrate plasma coated with HA, has a thermal conductivity of 6 W/mK. As noted above, the melt temperature of HA is 1650.degree. C. Further, the plasma temperature can be as high as 30,000.degree. C.

To ensure that the PEEK is not compromised, the temperature at the surface of the PEEK should not exceed 143.degree. C, the glass transition temperature (Tg) of PEEK. The glass transition temperature of a polymer, such as PEEK, is the point in which a transition occurs from a hard, glass state to a softer, rubber like state. At temperatures above PEEK Tg, molecular orientation of the polymer chains may occur, leading to changes in the polymer structure such as annealing or increased crystallization, which can affect the material properties. Temperatures above Tg may lead to oxidation of the PEEK, affecting its biocompatibility profile. Ensuring that the temperature at the PEEK surface does not go beyond Tg may be accomplished by adjusting various process parameters including, but not limited to, distance between the plasma torch and the substrate, step size of the plasma torch, traversing speed of the plasma torch and time between successive coatings.

Maintaining the surface temperature below the glass transition temperature is desirable because elevated temperatures can create alteration of the chemical structure of the PEEK and potentially biologically incompatible compounds or material properties. Maintenance of the chemical structure of PEEK also allows for known and reliable material properties acceptable for the regulatory approval process of medical devices.

The PEEK can be rotated during the plasma spray process, with the speed of rotation having an effect on the cooling rate. The PEEK implant can be attached to a shaft at attachment points in the implant. These attachment points may or may not be threaded. The shaft can then be rotated about the shaft axis. Pending the geometry of the implant, it may be necessary to rotate the PEEK at variable speeds in order to ensure uniform thickness of the coating.

The PEEK may also be cooled during the coating process or in between successive coatings by a suitable coolant. Suitable coolants generally consist of low temperature, non-reactive gases such as, but not limited to, carbon dioxide or nitrogen. The cooling will thus be adjusted as required to maintain the desired temperature at the surface of the PEEK.

Further, the hydroxyapatite coating has a crystallinity of at least about 45%. Contrary to prior art HA coated implants, in which the thinner the coating, the lower the crystallinity thereof, the HA coating of the implant body has a crystallinity of at least about 45%. In general, the HA coating comprises crystalline islands amongst amorphous domains. As the amorphous domains are resorbed by the adjacent bone in vivo, the crystalline islands are released. If the crystallinity of the coating is too low, premature delamination of the coating can occur. As a result, the crystalline islands can become phagocytized by macrophages, causing an acute inflammatory response. Therefore, as discussed earlier, the crystallinity is sufficient such that, as the bone is resorbed, delamination is minimized or prevented.

Crystallinity of the HA coating is affected by the annealing, or cooling rate of the HA after it has been deposited onto the PEEK substrate. Preferably, the HA coating is permitted to cool at a slow rate, thereby increasing the crystallinity of the coating. To further increase crystallinity, the temperature difference between the HA coating and the PEEK substrate must be accounted for so that the HA first contacting the PEEK can cool at an appropriate rate without overheating the outer surface of the PEEK substrate or requiring a pre-heated PEEK substrate outer surface to minimize the temperature differential. Traditionally, in HA coated titanium substrates, the implant can be reheated to recrystallize the HA coating after deposition. However, due to the lower glass transition and melting temperatures of PEEK, this is not an available option.

Any combination of techniques can be used to maintain the surface temperature of the PEEK below the glass transition temperature. Various methods can be used to obtain the HA coated PEEK devices of the instant invention, which provide an unaltered biocompatibility profile of the PEEK substrate, along with a coating thickness ranging from about 45 to about 100 micrometers, a coating crystallinity of at least 45%, and a bond strength between the coating and the substrate of at least 15 MPa. An exemplary process follows, but one skilled in the art will appreciate that any combination of these parameters could ensure the desired temperature at the surface of the PEEK:

TABLE 2
Plasma Spray Parameters

| | | |
|---|---|---|
| Stand off distance: | 5-20 | cm |
| Gun traversing speed: | 45-70 | cm/s |
| Step size: | 2-5 | mm |
| Primary gas flow: | 40-120 | l/m |
| Time between coats: | 10-30 | s |

HA coating of PEEK can be applied to any PEEK implant device, including, for example, the implant devices described in U.S. Pat. No. 7,001,433 to Songer et al. and U.S. patent application Ser. Nos. 10/692,468; 10/971,734; 11/856,565; 11/856,667; 11/750,612 and 11/259,403, all of which are hereby incorporated by reference as if reproduced in their entirety herein. A collection of exemplary PEEK VBRs and motion preservation devices that can have HA coatings applied thereon are shown in FIGS. 6-15.

The VBRs and motion preservation devices shown in FIGS. 6-15 can include both coated and uncoated PEEK substrate portions. The PEEK substrates are preferably coated along any surfaces which will come into contact with bone, particularly when in the final operative position. However, other portions of the implant body may not include a hydroxyapatite coat. In particular, portions which may be subjected to elevated levels of stress may remain uncoated. To minimize hydroxyapatite delamination or cracking, the spray coating process is preferably conducted so as to not coat these areas which may be subjected to high levels of stress. In particular, these areas may include tool engagement portions, rotating portions, and portions which may cut or penetrate the adjacent bone.

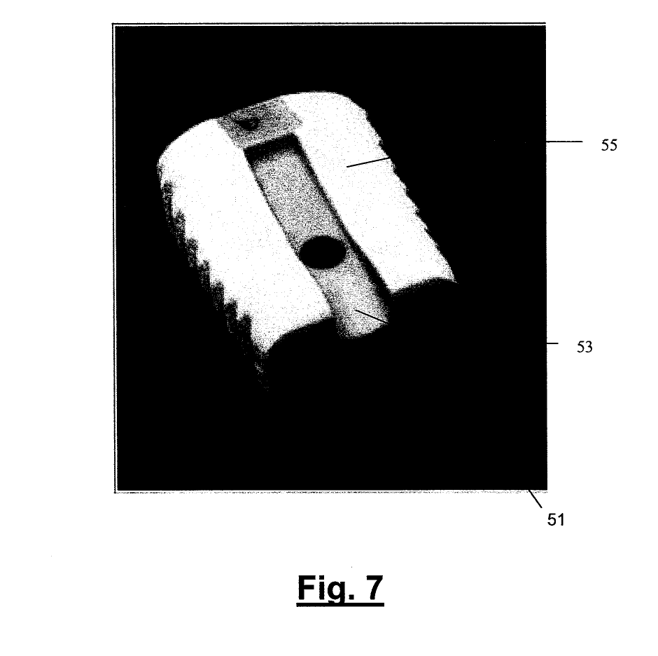

For example, the implants shown in FIGS. 7-10A, are configured to be rotatable along their longitudinal axes after implantation between adjacent vertebrae. In order to rotate the implants, a tool must engage the tool engagement portion and rotate the implant, which may result in increased stress at the point of engagement between the tool and the tool engagement portion. In order to maintain the integrity of the HA coating and maximize the strength of the implant, particularly along the tool engagement portions, the implants include coated portions and uncoated portions, the uncoated portions including the tool engagement portion of the implant.

FIG. 7 shows an implant 51 (such as described in U.S. patent application Ser. Nos. 11/259,403 and 11/750,612) with an HA coating having been applied. The HA coated surface 55 has a whiter color than the PEEK substrate 53. The PEEK substrate 53 is visible at the tool attachment points of the implant and corresponds to the attachment points when the plasma spray process is applied to the implant 51.

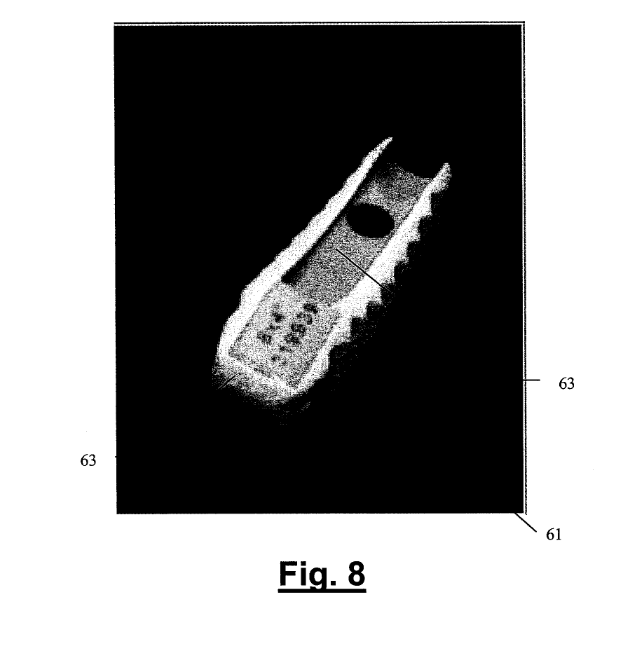

FIG. 8 shows an alternate configuration of the above implant, implant 61 having an HA coating. The implant 61 is configured to provide structural support to adjacent bone and allow room for the packing of bone void filler. The HA coated contact surface 65 is white in color compared to the gray color of the PEEK substrate 63. The PEEK substrate 63 corresponds to the area used as mounting points when the plasma spray process is applied to the implant 61.

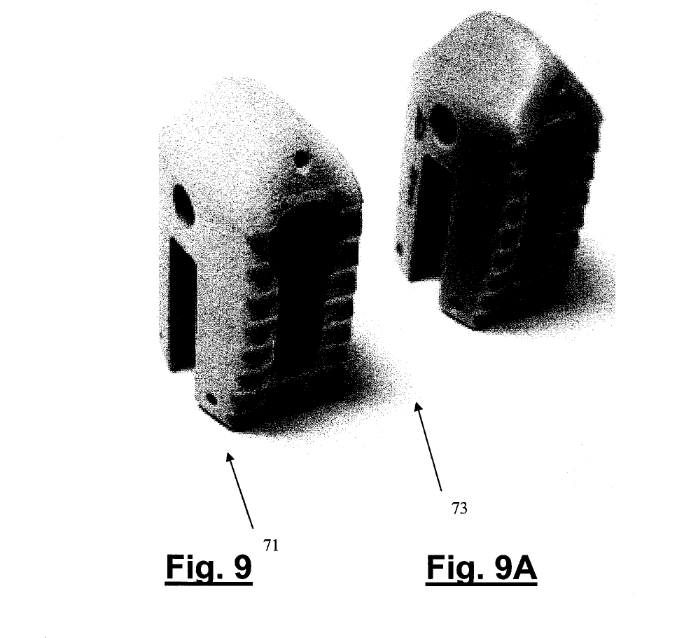

FIGS. 9 and 9A show an HA coated implant 71 and an uncoated implant 73 (such as described in U.S. patent application Ser. Nos. 11/259,403 and 11/750,612). The HA coated bullet tip implant 71 is a white in color with a visibly porous surface compared to the smooth surface color of the PEEK only bullet tip implant 73.

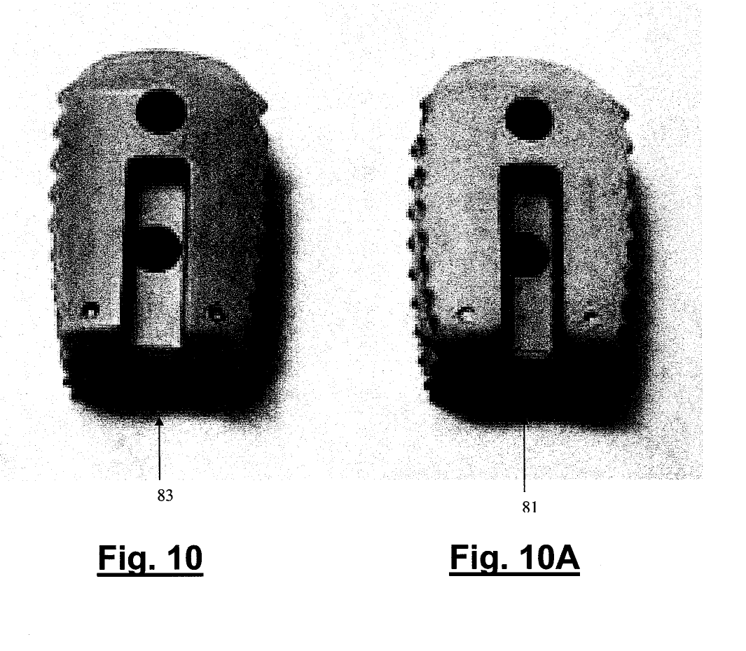

FIGS. 10 and 10A show a HA coated bullet tip implant 81 and an uncoated bullet tip implant 83. The implant 83 is configured to provide a multifunctional implant for a variety of bone support needs. The HA coated bullet tip implant 81 advantageously maintains the multifunctional configuration but with increased bioactivity due to the HA coating.

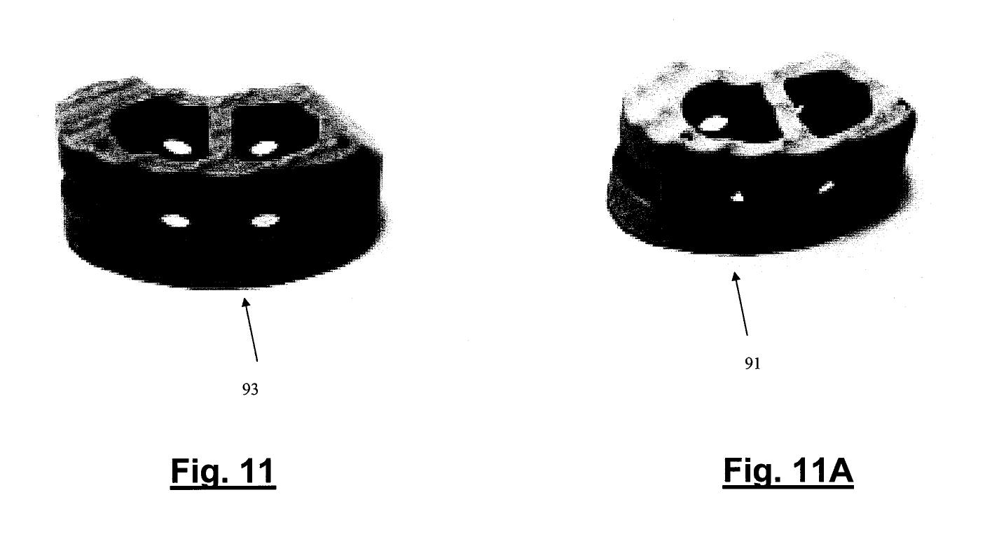

FIGS. 11 and 11A show the HA coated implant 91 and an uncoated implant 93 (such as described in U.S. patent application Ser. No. 11/750,612). The HA coated implant 91 is a white in color compared to the gray color of the uncoated implant 93.

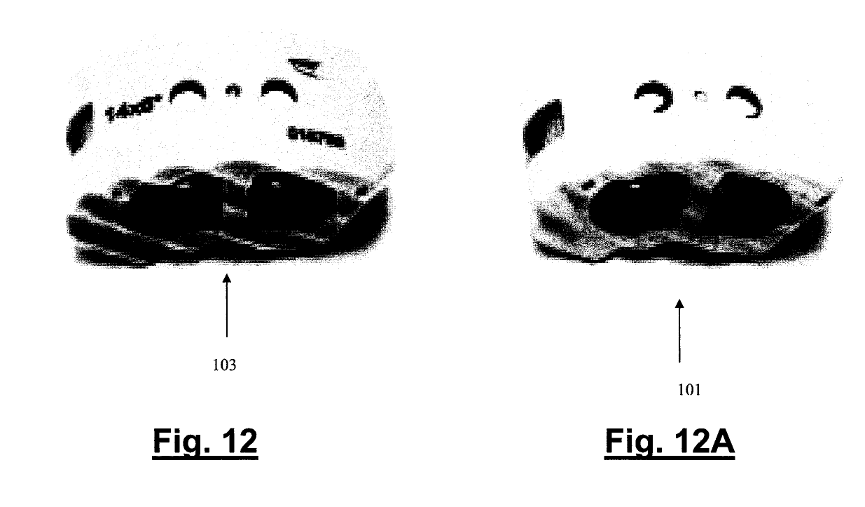

FIGS. 12 and 12A show an HA coated implant 101 and an uncoated implant 103 (such as described in U.S. patent application Ser. No. 11/750,612). The implant 103 is configured to provide structural support that conforms to the vertebrae and provides for the packing of bone void filler. The HA coated implant 101 maintains the conforming structural shape but with increased bioactivity due to the HA coating.

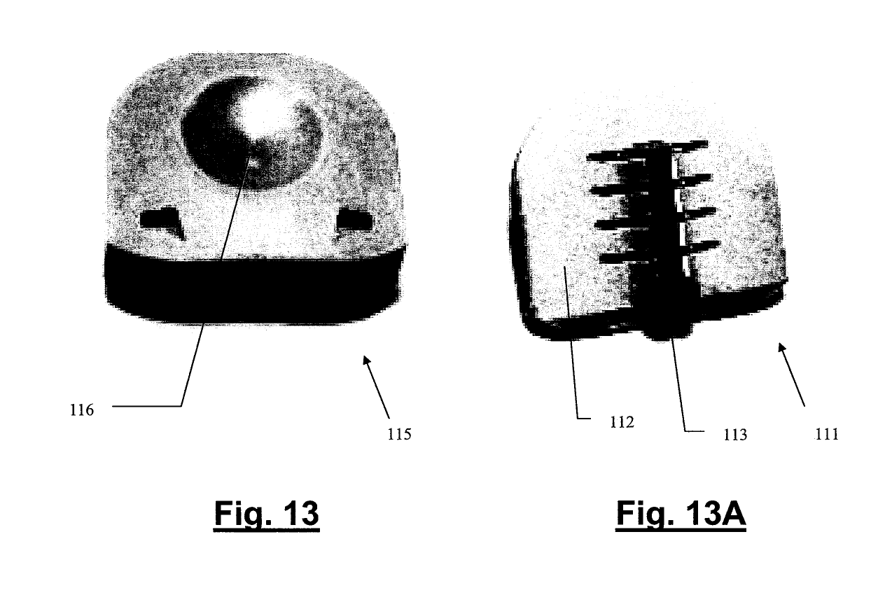

FIGS. 13 and 13A show the top surfaces of the top portion 111 and a bottom portion 115 of a motion preservation implant (such as described in U.S. Pat. No. 7,001,433 to Songer et al. and U.S. patent application Ser. Nos. 10/692,468; 10/971,734; 11/856,565; and 11/856,667). The top portion 111 is shown with an HA coating 112 having been applied to the top surface and an uncoated metallic cam 113. The bottom portion 115 is shown with a PEEK coating visible on the top surface 116. The top portion 111 and bottom portion 115 form a ball and socket arrangement for articulation. The top surface 116 of the bottom portion 115 forms the socket.

The metallic cam 113 is not coated due to the stresses applied to the cam 113. When the cam 113, in particular the cutting edges thereof, engages and enters into the adjacent bone, the cam 113 is subject to increased stress. Such stress could damage any hydroxyapatite coating of the cam 113, such as by delamination, which would be undesirable.

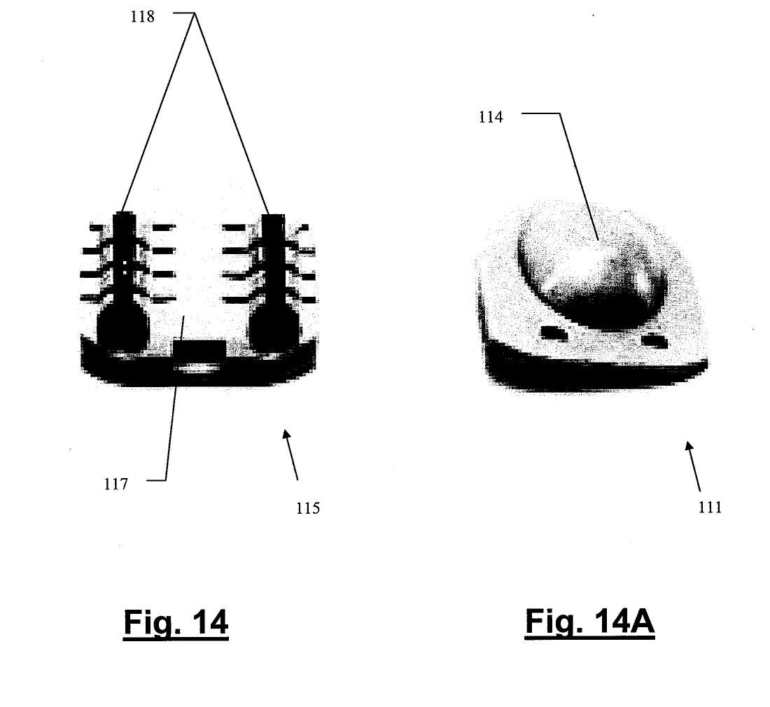

FIGS. 14 and 14A show the bottom surfaces of the top portion 111 and the bottom portion 115 of the implant of FIG. 13. The top portion 111 is shown with the PEEK bottom surface 114 visible for the ball of the ball and socket arrangement. The bottom portion 115 of bottom surface 117 is also shown. The bottom surface 177, which comes into contact with bone, is HA coated and is embedded into the bone with the cams 118. The cams 118 are metallic and are anodized and are not coated, as discussed above.

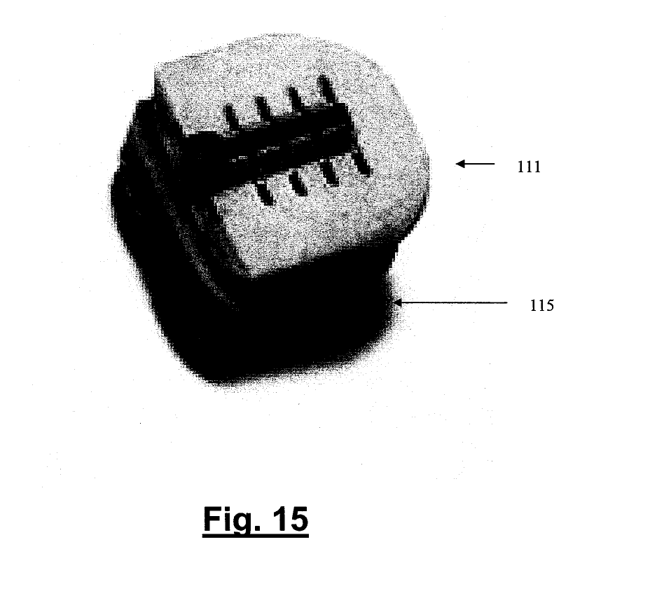

FIG. 15 shows the assembly of the top portion 111 and bottom portion 115 in the ball and socket arrangement. The implant is configured to provide structural support to adjacent bone and allow for some mobility in the spine of the patient.

It is foreseeable that these inventions could provide uses beyond inter-body fusion or motion preservation devices and be easily incorporated into orthopedic devices generally for mending all types of broken bones and injured joints. In particular, the implants as shown in FIGS. 13-15, having an articulating ball and socket arrangement, are particularly appropriate as a joint replacement devices in which the HA coated surfaces could be used to securely fuse the implant to the remaining bone with assistance of the cams.

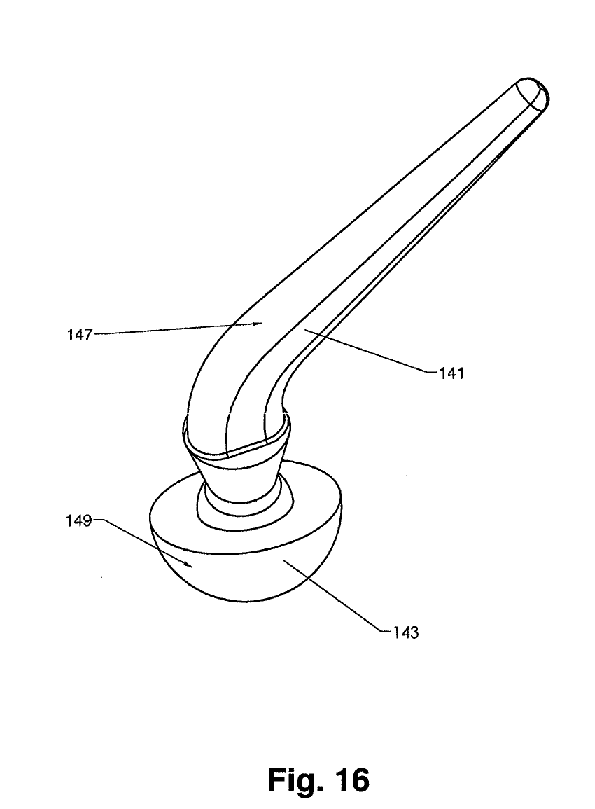

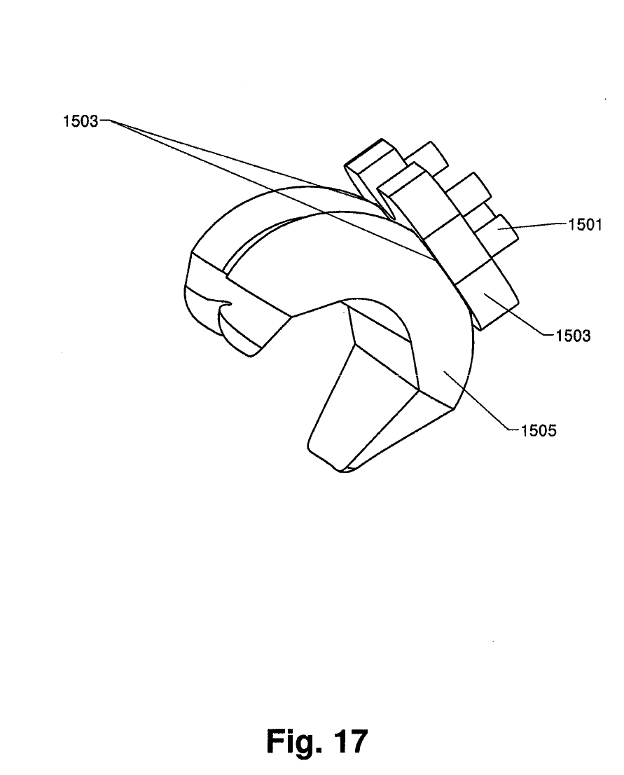

Examples of the motion preservation concepts being used for other types of implants are shown in FIGS. 16 and 17. FIG. 16 shows a hip replacement implant with an articulating ball and socket arrangement. The implant is comprised of two PEEK components; the ball portion 141 and the socket portion 143. The ball portion 141 fits within the socket portion 143. The ball portion 141 and socket portion 143 are configured to articulate in contact with one another without creating substantial wear debris. The ball portion surface 147 and bone bearing surface 149 are preferably coated with HA in accordance with the present invention.

FIG. 17 shows a knee replacement implant that is able to articulate by a top portion 1503 traveling along a curved surface 1506 of a bearing member 1505, the top portion 1503 including sockets 1507 configured to receive and engage the curved surface 1506. The top portion 1503 provides a bearing surface for both the lower portion 1505 and the femur (i.e. upper leg bone) of the patient which attaches with pins 1501. The lower member 1505 provides a bearing surface 1506 to the upper portion 1503 and also attaches to the tibia (i.e. the lower leg bone) of the patient along a generally curved engaging surface 1509. Preferably, at least the pins 1501 and the curved engaging surface 1509 are coated with HA in accordance with the present invention. Further, the top portion 1503 can be coated with HA in accordance with the present invention.

Several techniques are available to those skilled in the art for determining whether a polymer has been thermally degraded by exposure to an increase in temperature that may be detrimental to the PEEK or other polymers. Differential Scanning Calorimetry (DSC) and Attenuated Total Reflected-Fourier Transform InfraRed Spectroscopy (ATR-FTIR) are commonly used. DSC is a thermal analytical technique in which the difference in the amount of heat required to increase the temperature of a reference and sample are measured as a function of temperature. Both the sample and reference are maintained at nearly the same temperature throughout the analysis. Differential scanning calorimetry can be used to measure a number of characteristic properties of a sample. Using this technique it is possible to observe fusion and crystallization events as well as glass transition temperatures. DSC can also be used to study oxidation, as well as other chemical reactions.

Glass transitions may occur as the temperature of the sample is increased. These transitions appear as a step in the baseline of the recorded DSC signal. This is due to the sample undergoing a change in heat capacity. At some point the molecules in the sample may obtain enough freedom of motion to spontaneously arrange themselves into a crystalline form. This is known as the crystallization temperature (Tc). This transition from amorphous solid to crystalline solid is an exothermic process, and results in a peak in the DSC signal. As the temperature increases the sample eventually reaches its melting temperature (Tm). The melting process results in an endothermic peak in the DSC curve. The ability to determine transition temperatures and enthalpies makes DSC an invaluable tool in producing phase diagrams for polymers. Any oxidation that occurs in the sample is observed as a deviation in the baseline.

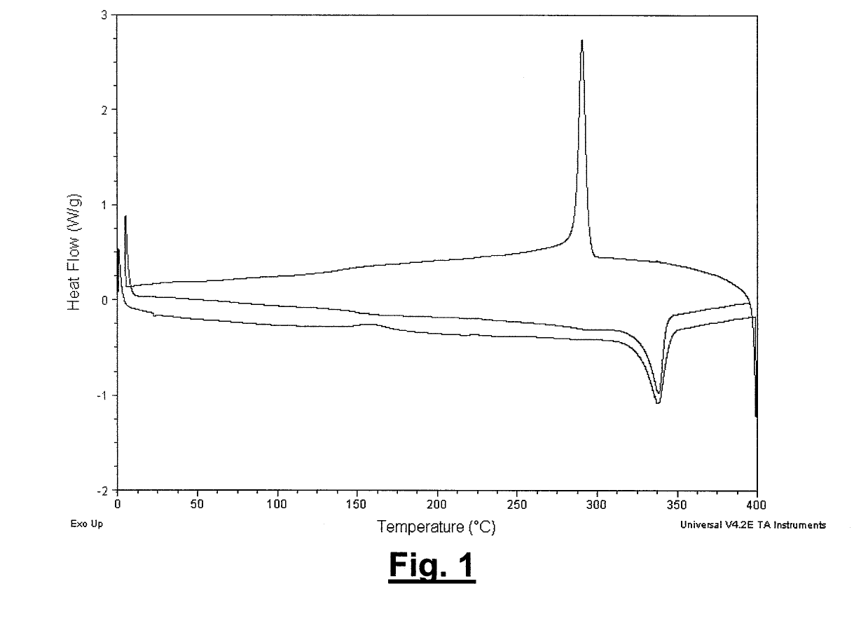

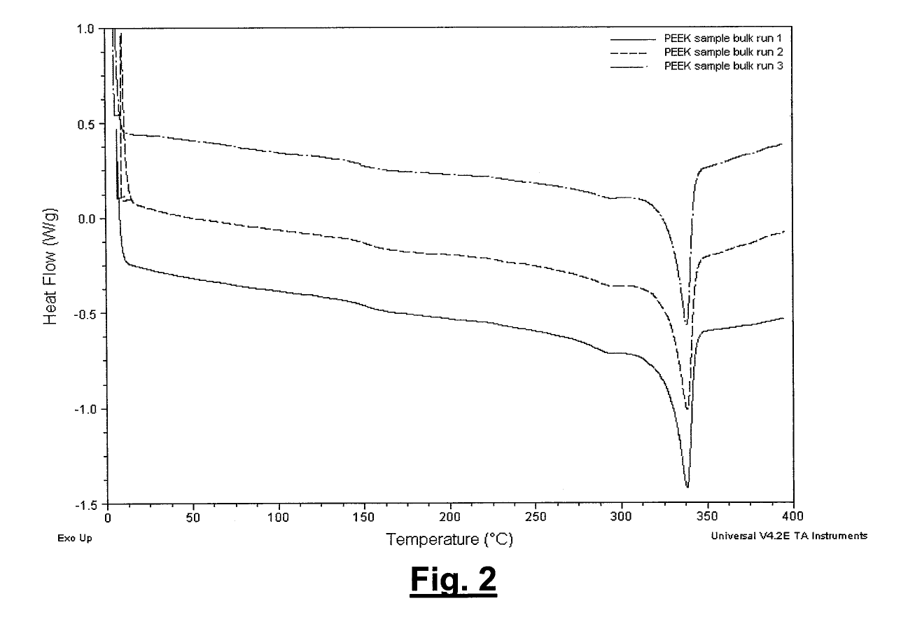

For the present invention, a razor blade was used to remove sections from the component at the surface and in the bulk of the sample, which was then weighed on a precision balance. The balance is routinely calibrated with precision masses. The sample was then crimped between an aluminum pan and lid, and placed in a cell. An empty aluminum pan and lid were used as a reference. The sample was run through a heat/cool/heat cycle, over a temperature range of 0-400° C. The sample was heated and cooled at a rate of 20° C. per minute. FIG. 1 and FIG. 2 give evidence of the heat/cool/heat cycle results to support that the present invention does not result in any deviation from the baseline. The traces are vertically offset for clarity. Measurements were taken at the surface and in the bulk of both materials. Table 3 provides a summary of the data gathered through the DSC analysis from the second heating cycle of FIG. 2.

TABLE 3
DSC Second Heating Cycle Analysis

| | | Tm, | Tm, | | |
|---|---|---|---|---|---|
| | | onset | peak | ΔH | |
| Sample | | (° C.) | (° C.) | (J/g) | Tg ((° C.) |
| Surface | Run 1 | 324.52 | 337.39 | 67.79 | 148.90 |
| Surface | Run 2 | 324.80 | 336.78 | 65.03 | 144.88 |
| Surface | Run 3 | 325.30 | 337.00 | 61.64 | 149.58 |
| | Average | 324.87 | 337.06 | 64.82 | 147.79 |
| | St. Dev. | 0.4 | 0.31 | 3.08 | 2.54 |
| Bulk | Run 1 | 325.59 | 338.12 | 56.28 | 151.12 |
| Bulk | Run 2 | 325.80 | 338.45 | 62.52 | 151.84 |
| Bulk | Run 3 | 326.04 | 338.39 | 59.32 | 148.90 |
| | Average | 325.81 | 338.32 | 59.37 | 150.62 |
| | St. Dev. | 0.23 | 0.18 | 3.12 | 153 |

Infrared (IR) spectroscopy is a chemical analytical technique that provides an analysis 0.5-5.0 μm below the surface, and measures the infrared intensity versus wavelength (wave number) of light. Based upon the wave number, infrared light can be categorized as far infrared (4˜400 cm-1), mid infrared (400˜4,000 cm-1) and near infrared (4,000˜14,000 cm-1).

Infrared spectroscopy detects the vibration characteristics of chemical functional groups in a sample. When an infrared light interacts with the matter, chemical bonds will stretch, contract and bend. As a result, a chemical functional group tends to adsorb infrared radiation in a specific wave number range regardless of the structure of the rest of the molecule. For example, the C═O stretch of a carbonyl group appears at around 1700 cm-1 in a variety of molecules. Hence, the correlation of the band wave number position with the chemical structure is used to identify a functional group in a sample. The wave number positions where functional groups adsorb are consistent, despite the effect of temperature, pressure, sampling, or change in the molecule structure in other parts of the molecules. Thus the presence of specific functional groups can be monitored by these types of infrared bands, which are called group wave numbers.

A Fourier Transform Infrared (FTIR) spectrometer obtains infrared spectra by first collecting an interferogram of a sample signal with an interferometer, which measures all of infrared frequencies simultaneously. An FTIR spectrometer acquires and digitizes the interferogram, performs the FT function, and outputs the spectrum.

Attenuated total reflection infrared (ATR-FTIR) spectroscopy is used for analysis of the surface of materials. It is also suitable for characterization of materials which are either too thick or too strong absorbing to be analyzed by transmission spectroscopy. For the bulk material or thick film, no sample preparation is required for ATR analysis. For the attenuated total reflection infrared (ATR-FTIR) spectroscopy, the infrared radiation is passed through an infrared transmitting crystal with a high refractive index, allowing the radiation to reflect within the ATR element several times.

For the present invention, the infrared spectrum for the PEEK sample was obtained by contacting the surface of the sample with a Germanium crystal, using light pressure. Scans of the surface were taken at three locations on the surface of each sample to get a better representation of the surface and to ensure that the surface was chemically uniform. There were no changes evident in the molecular structure of the sample, indicating no molecular or functional group changes as shown in FIG. 3.

While there have been illustrated and described particular embodiments of the present invention, it will be appreciated that numerous changes and modifications will occur to those skilled in the art which fall within the true spirit and scope of the present invention.

What is claimed is:

1. An implant device for supporting bone comprising:
an implant body comprising a biocompatible polymer material;
a coating of the implant body comprising hydroxyapatite;
a Ca/P ratio of the coating ranging from about 1.65 to about 1.76;
a coating crystallinity greater than about 45% and less than about 5% of other crystalline phases;
a mechanical bond strength between the implant body and the coating of at least about 15 MPa; and
a coating thickness of about 45 to about 100 micrometers, wherein the coating resists delamination during expected in vivo stress.

2. The implant device of claim 1 wherein the implant body comprises poly-ether-ether-ketone.

3. The implant device of claim 1 wherein the coating is plasma sprayed on the implant body.

4. The implant device of claim 1 wherein the implant body is biocompatible along an outer surface thereof.

5. The implant device of claim 1 wherein the coating thickness ranges from about 45 to about 80 micrometers.

6. The implant device of claim 1 including an uncoated portion of the implant body.

7. A weight-bearing implant for being implanted within a weight-bearing joint, comprising:
an implant body comprising poly-ether-ether-ketone and configured to fit at least partially within a weight bearing joint:
a deployable securing mechanism connected to the implant body;
a camming surface of the securing mechanism;
a corresponding camming surface of the implant body, the camming surfaces configured to resist deployment and retraction of the deployable securing mechanism;
a biocompatible, resorbable coating of hydroxyapatite mechanically adhered to the implant body;
a bond strength of at least 15 MPa between the coating and the implant body;
a Ca/P ratio ranging from about 1.65 to about 1.76;
a coating thickness ranging from about 45 to about 100 micrometers; and
a coating crystallinity of at least about 45%,
wherein the coating further comprises less than about 5% of other crystalline phases.

8. The implant device of claim 7 wherein the implant body is biocompatible along an outer surface thereof.

9. The implant device of claim 7 wherein the coating thickness ranges from about 45 to about 80 micrometers.
